(19) United States
(12) Patent Application Publication
(10) Pub. No.: US 2004/0167318 A1
(43) Pub. Date: Aug. 26, 2004

(54) Process for extracting collagen from marine invertebrates

(76) Inventor: Bhanumathy Manickavasagam, St. Lucia (AU)

Correspondence Address: EITAN, PEARL, LATZER & COHEN ZEDEK LLP, 10 ROCKEFELLER PLAZA, SUITE 1001, NEW YORK, NY, 10020, US

(21) Appl. No.: 10/480,829

(22) PCT Filed: Jun. 14, 2001
(86) PCT No.: PCT/AU01/00708

(51) Int. Cl. C07K 14/78

(52) U.S. Cl. 530/356

(57) ABSTRACT

A process for isolating a collagen-derived protein fraction from a marine invertebrate, comprising the steps of: 1) preparing a collagen-containing portion of said marine invertebrate for extraction; 2) treating the collagen-containing portion with a weak acid solution in order to solubilise a collagen-derived protein fraction; and 3) collecting the collagen-derived protein fraction.

### TECHNICAL FIELD

The present invention is concerned with a process for obtaining native collagen through extraction from marine invertebrates. The collagen obtained is both a novel protein and an alternative product to land animal collagen due to the current concerns about Bovine Spongiform Encephalopathy (BSE) or Mad Cow Disease. It also relates to a novel process for isolating a collagen-derived protein fraction, such as collagen itself or gelatin.

### BACKGROUND ART

BSE is an extremely serious disease of cattle, considered to originate from infected meat and bone meal in cattle feed concentrates. BSE is transmissible in cattle, and was first identified in United Kingdom in 1986. It is invariably fatal. There is no treatment and it is difficult to detect. Recent research indicates that humans who eat infected meat could develop Creutzfeldt-Jacob Disease (CID), the human equivalent of the cattle disease. At least 10 CID patients in Britain are believed to have contracted the disease from eating beef. Most people who develop CID are aged between 50 and 70.

Currently the culling of the cattle is of primary importance in the United Kingdom and Europe to safeguard the herd. Nevertheless, BSE poses a significant threat to the future supply of bovine meat and dairy products for the human and animal food chains, and to the supply of important bovine by-products used in the pharmaceutical, medical and cosmetic industries. Presently, the manufacturers of pharmaceuticals across Japan, UK and Europe and other countries have stopped using British beef and beef products in the manufacture of pharmaceuticals and medicines as well as cosmetics products to prevent the spread of “Mad Cow” disease to humans. Also imports of medicine and cosmetics containing substances from British cows have stopped.

The most widely used beef product is collagen. Collagen is a fibrous protein which comprises most of the white fibre in the connective tissues of mammals, particularly the skin, tendon, bone and muscles. A number of different vertebrate collagen have been identified, up to 19 groups so far have been identified in vertebrates (Prockop and Kivirikko, 1995) of which type I, II and III represent the most widely distributed species. Collagen comprises about 30% of the total organic matter in mammals and nearly 60% of the protein content. Collagen is deposited rapidly during periods of rapid growth, and its rate of synthesis declines with age, particularly in tissues that undergo little remodeling.

The collagen molecule is built from three peptide chains which are helical in conformation. The helix extends through 1014 residues per chain (Hoffmann et al 1980). At the end of the triple helical domain, short non-helical chains, namely telopeptides, having a non-repeating sequence and spanning from 9 to 25 residues, extend beyond the triple helix from both ends of each chain (Hoffman et al, 1980). The telopeptide portions of native collagen are believed to be the major sites of its immunogenicity and have been shown to play a crucial role in directing fibrillogenesis (Helseth and Veis 1981). The length of the helix and the nature and size of nonhelical portions of the molecule vary from type to type. If the triple helical structure of the collagen molecule is destroyed by heat, the properties of the polypeptides change entirely in spite of having the same chemical composition.

In skin, collagen exists as fibres which are woven into networks constituting fibre bundles, the fibres being maintained in the bundle by interfibrillar cement. Collagen fibrils typically have a length of about 2 mm while the fibres are naturally much longer and of greater diameter.

Vertebrate collagen has a molecular weight of 300,000 Daltons. Each strand of the triple helix has a molecular weight of approximately 100,000 Daltons and assumes a left-handed helix configuration (Lehninger 1975). Most vertebrate collagens present in skin, tendon, muscle, and bone are composed of two identical and one different α chains denoted by [(α1)₂ α2] (Piez et al. 1963; Lewis and Piez, 1964; Miller et al, 1967; McClain et al. 1970) except for codfish skin and chick bone collagen which contains three different chains [(α1) (α2) (α3)] (Piez, 1965; Francois and Glincher 1967). Cartilage collagen has in addition to molecules of chain composition [(α1)₂ α2], another type of molecule which is composed of three identical chains, [α1 (II)₃] [SHOULD THIS BE α1 (II)₃?] (Miller 1971; Trelstad 1970). The α1 (II) chain is apparently different from the α1 chain, which is designated α1(I) only when compared to α1 (II), in its high content of glycosylated hydroxylysines. The collagen present in basement membranes (Kefalides, 1971) and sea anemone body wall (Katzman and Kang 1972) have also been confirmed to consist of identical α chains.

Collagen is the only mammalian protein containing large amounts of hydroxyproline and it is extraordinarily rich in glycine (approximately 30%) and proline. The hydroxyproline is essential for the formation of hydrogen-bonded water-bridges through the hydroxyl group and the peptide chain, thereby stabilising the triple helix. In soluble collagen the inter-molecular bonds have been cleaved, but leaving the triple helices intact.

Collagen type I, especially bovine skin collagen, has been utilised in foods and beverages, cosmetics and medical materials. Purified adult bovine collagen is used in a variety of medical devices, including hemostats, corneal shields, and for soft tissue augmentation. Collagen gels are often intermediates in the preparation of these devices and, in some cases, the gels represent the final medical products. There are also lyophilised collagen masks or face-packs intended for use on the skin, both for therapeutic and cosmetic purposes. Purified calf skin collagen is an important biomaterial used in several devices as prostheses, artificial tissues, material for construction of artificial organs and as a drug carrier because the collagen molecule is non-toxic toward an organism and has a high mechanical strength. It is also useful in cosmetic compositions for the same reason.

In the biomedical field natural fibres are used in sutures and ligatures. A ligature is a thread used to tie off a bleeding vessel, while a suture is used to sew up a wound. The wound may be internal or it may be exposed. The sutures used for closing an internal wound are less easily removed. Thus an absorbable (or biodegradable) material offers a distinct advantage.

As the collagen becomes increasingly cross-linked it also becomes less hygroscopic. One of the effects of ageing in mammals is an increase in the cross-linking of collagen molecules. As cross-linking increases, it becomes more and more difficult to extract tropocollagen from mammalian sources. Increasing cross-linking is related with increasing biological age of the collagen. Uncross-linked tropocollagen has been used in cosmetics because of its association with unwrinkled skin.

Vertebrate collagen generally has to be purified extensively to remove all non-collagenous, contaminating structures. The final product of most-collagen isolation and purification procedures, which consist mainly of enzymatic degradation of the non-collageneous component of connective tissue, are monomeric collagen molecules. When these rods are reconstituted into films, membranes, or sponges they will contribute very little to the mechanical strength of the final structure. It would be desirable in a purification procedure to preserve the natural structure of collagen fibres and fibrils. Due to the length (2-10 cm) and thickness (40 μm) of these highly pure collagen fibres, they can be further processed into threads, sutures or non-woven fleece layers, and may be knitted or woven.

Two methods have been applied to solubilise the highly cross-linked collagen tissue in vertebrates. These are (1) digestion with proteolytic enzymes and (2) treatment with alkali.

Proteolytic digestion with enzymes such as pepsin is often used because of the relative ease with which the cross-links in collagen may be broken. Pepsin is the most commonly used enzyme because it is available in pure form from commercial sources and can be employed in an acidic solvent in which the monomer molecules readily dissolve. Although limited proteolysis with pepsin has been extremely useful in preparing relatively large amounts of the various collagens in essentially monomeric form from a number of animal and human tissues, the procedure has its limitations. For example, the molecules are obtained with altered nonhelical extremities, and this effectively precludes subsequent studies designed to evaluate the structure and function of these regions. Furthermore, since enzyme-solubilised collagen is rich in monomeric collagen but without telopeptides, collagen fibril reconstruction is greatly inhibited and reconstructed fibrils show low thermal stability as compared with soluble collagen with telopeptides.

Collagen hydrolysates prepared from native collagen by enzymatic hydrolysis to form peptides exhibit molecular weights in the range of 1,000 to 10,000 Daltons. In vertebrate tissue the process takes at least 2-3 days for complete extraction at 4° C.

Alkaline treatment is usually performed by immersing collagenous tissues in a 2-5% sodium hydroxide solution containing sodium sulphate and amines as a stabiliser and a nucleophile, respectively, at 4-20° C. for several days. The tissue is then further treated with acid. It is a time-consuming process which takes up to several months, depending on environmental temperatures. Traditionally bovine hide has been conditioned by an alkaline liming process, which takes many weeks. The alkaline treatment modifies the protein by partly removing amine and amide groups. Most of the swelling and hydrolysis of amide groups occurs during the early stages of liming, and there is noticeable evolution of ammonia as the collagen isoelectric point falls near pH 5.

Gelatin is another very important biopolymer that has found widespread use in the food, pharmaceutical and photographic industries over the years. Traditionally it occurs as a transparent dessert jelly, but is widely used in confectionery, jellied meats and chilled dairy products.

Gelatin is a protein derived from collagen. The source and type of collagen will influence the properties of the resulting gelatin. The amino acid content and sequence varies from one source to another but always consists of large amounts of proline, hydroxyproline and glycine. Since most of the commercial gelatins are obtained from either pigskin or cowhide, there has been considerable interest in pursuing alternative substitutes. This has especially been the case since the recent BSE (bovine spongiform encephalopathy) crisis.

When collagen is heated at a certain temperature the collagen molecule undergoes a helix coil transition. The helix unfolds and the collagen becomes more readily soluble. The temperature at which this occurs depends upon the amount of proline and hydroxyproline in the α chain, and this temperature is the point of denaturing. For deep cold water fish collagen, this temperature is approximately 15° C. while for bovine collagen it is approximately 40° C. At a certain temperature the collagen in the raw skin will relax and the skin will shrink (shrinkage temperature). The amount of imino acids, praline and hydroxyproline, determines the shrinkage temperature and the denaturing temperature.

From vertebrates, the raw materials used for the manufacture of gelatin are pigskin, cattle hides, and cattle bones. The processing of gelatin from these raw materials involves numerous steps and the yields are low. Severe processing is required to solubilise gelatins from stable highly cross-linked ossein (crushed, acid-demineralized and degreased bone) and cattle hide. Gelatins derived from these sources are almost fully deamidated and have isoelectric points close to pH 5.

From vertebrates, the extraction of gelatin depends upon both dissolving and hydrolysing the denatured skin. The gelatin may retain some covalent bonds between alpha chains, which would give rise to multiples of single chain lengths of 95,000 Daltons.

The melting and gelling temperature of gelatin has been found to correlate with the proportion of the imino acids, proline and hydroxyproline in the original collagen (Veis 1964). This is typically approximately 24% for mammals and 16-18% for most fish species.

Thus the conversion of insoluble collagen to soluble gelatin constitutes the essential transformation in gelatin manufacture. The properties of the gelatin depend to a great extent on the raw material employed, on the decomposition process selected, and especially on the reaction conditions during decomposition, extraction, and drying.

In general, there is no satisfactory way to purify native insoluble collagen fibrils, especially from a tissue in which the collagen is highly cross-linked. However, the present invention provides a means by which native collagen may be obtained, as well as a novel process for isolating a collagen-derived protein fraction.

### DISCLOSURE OF THE INVENTION

According to a first aspect of the present invention there is provided a process for isolating a collagen-derived protein fraction from a marine invertebrate, comprising the steps of:

1) preparing a collagen-containing portion of said marine invertebrate for extraction;

2) treating the collagen-containing portion with a weak acid solution in order to solubilise a collagen-derived protein fraction; and

3) collecting the collagen-derived protein fraction.

If the temperature is maintained below that at which collagen converts to gelatin, native collagen is collected. However, if the collagen-derived protein fraction is extracted at the temperature above that at which collagen converts to gelatin, gelatin is collected. In order to collect native collagen, the temperature should preferably be maintained below 25° C., and more preferably below 4° C. When gelatin is to be collected the temperature for the extraction is preferably 40° C. or above.

In either process it is preferable that the weak acid solution is an acetic acid solution, typically a 0.1M to 1M solution, preferably a 0.5M solution. A weak acid is one with a dissociation constant between 1.0×10⁻⁵ and 1.0×10⁻² in aqueous solution and so is predominantly un-ionised, and these may be readily identified by the person skilled in the art but include lactic, butyric, formic, propionic and citric acids.

In isolating native collagen it is preferable that the pH of the native collagen-containing weak acid solution be adjusted from time to time. Typically a strong acid such as hydrochloric acid is added. A 1.0N hydrochloric acid solution is typical, and is ideally used in small amounts to adjust the pH to around 3.5.

Advantageously, the pH adjustment takes place after the collagen-containing portion has been in contact with the weak acid solution for two to twelve hours. Native collagen may then be collected after a further period, typically 6 to 24 hours, of contact. There may be an additional period of contact between the collagen-containing material and the weak acid solution after this collection, followed by a further collection of additional native collagen. This cycle may continue to be followed until all extractable collagen has been collected, and there may be pH adjustments as appropriate throughout the process.

Typically the native collagen is collected by salting out the protein, but any suitable means for collecting the product may be used and these will be well known to the person skilled in the art.

Advantageously, the collagen-containing portion is spun down in a centrifuge and native collagen is precipitated from the supernatant. Additional collagen may be extracted from the pellet, if desired. In a typical collection process from the supernatant, sufficient sodium chloride, typically in solid form, is added to bring the supernatant to 0.3M sodium chloride.

The native collagen may be purified in a further purification step. Suitable means for purification of proteins are well understood by the person skilled in the art. Typically, the native collagen may be prepared as a white lyophilisate, but could also be prepared as a paste or in any other suitable form.

Advantageously, the salt used to precipitate the native collagen is removed by dialysis against de-ionised water. The native collagen may also be dialysed against a weak acid solution in order to adjust the pH of the solution inside the dialysis bag until it reaches a desirable pH, typically pH 3.5, prior to freeze-drying.

Typically the weak acid solution is subjected to some form of agitation during the process described above, whether it be gelatin or native collagen which is the product. Preferably, the collagen-containing portion is suspended in the weak acid solution, and the suspension is stirred in order to ensure good yield and high product quality. However, if stirring is not effected some product of lesser quality may still be obtained. The freeze-dried material obtained is cream coloured and rubbery with poor aqueous solubility, as compared to the usual product which consists of white, crisp fibres which are readily soluble.

Typically the marine invertebrate is prepared for extraction by mechanical disruption of the collagen-containing portion.

Advantageously, the collagen-containing portion is muscle tissue, which has preferably had pigment removed therefrom. This may be achieved by soaking the intact muscle tissue in a weak acid solution. The weak acid solution is typically an acetic acid solution, preferably a 0.2M solution.

In a particularly preferred embodiment of the invention, the marine invertebrate is abalone. Preferably the abalone is a commercial species such as the black-lip abalone, Haliotis ruber, the brown-lip abalone Haliotis conicopora and the green-lip abalone, Haliotis laevigata, or Roe's abalone, Haliotis roei.

According to a second aspect of the present invention there is provided a process for obtaining native collagen, comprising the steps of:

1) providing a marine invertebrate from which collagen may be extracted;

2) preparing a collagen-containing portion of the marine invertebrate for extraction;

3) treating the collagen-containing portion with a weak acid solution in order to solubilise a collagen fraction; and

4) collecting the native collagen.

The process of de-pigmenting the marine invertebrate is itself novel and constitutes a part of the present invention.

Accordingly, in a third aspect of the present invention there is provided a process of de-pigmenting a marine invertebrate having undesirable pigmentation, comprising the steps of:

1) isolating the food portion of the marine invertebrate; and

2) contacting the food portion with a weak acid solution in order to extract the pigment.

Preferably, the food portion is the muscle tissue of the marine invertebrate, which is typically abalone. As indicated above, the food portion may be soaked in the weak acid solution, which is typically a 0.2M acetic acid solution.

The present invention allows for the extraction of a collagen which is itself a novel product. The product of the processes described above therefore form part of the invention also. In addition, there is some characterising data for the polypeptide.

Accordingly, in a fourth aspect of the present invention there is provided an isolated polypeptide, said polypeptide being the α1 chain of type I abalone collagen, said polypeptide having a molecular weight of approximately 123.9 KD.

According to a fifth aspect of the present invention there is provided an isolated polypeptide, said polypeptide being the α2 chain of type I abalone collagen, said polypeptide having a molecular weight of approximately 110.6 KD.

Moreover, the α1 and α2 chains described above may be combined in order to produce a novel type I abalone collagen having the amino acid composition laid out in Table 8.

The polypeptides of the present invention are proposed for use in place of collagen isolated from land vertebrates or gelatin prepared from the collagen of land vertebrates. In particular, collagen may be used as a cosmetic ingredient, in the form of injectable collagen, in biomedical devices, as a pharmaceutical substance, in food products and beverages. The gelatin is useful at least in the form of edible gelatin and as a floculating agent in beverages, in industrial uses such as the manufacture of PVC pipes, glue and carbonless paper, as photographic gelatin for emulsion formulation, as a capsule coating for pharmaceuticals and as an ingredient of cosmetics. In addition, native collagen as described above may be used in the preparation of gelatin. This process involves providing native collagen, heating the native collagen to a temperature sufficient for conversion to gelatin to be effected, which is typically a temperature of at least 40° C.

### BRIEF DESCRIPTION OF THE DRAWINGS

Preferred embodiments of the invention will now be described by way of illustration only with reference to the accompanying drawings, in which:

FIG. 1 is a cross-sectional view of the abalone muscle showing

(A) dorsal surface of foot (epipodium)

(B) hard part of foot (pedal sole)

(C) soft part of foot (pedal sole)

(D) middle part of adductor (columellar) muscle

(E) upper part of adductor (columellar) muscle;

FIG. 2 is an SDS-PAGE gel of the various native abalone collagen fibrils (Parts A, B, C, D, D* and E); which are located in the following lanes:

Lane 1—molecular weight standard

2—Part A

3—Part B

5—Part C

7—Part D

8—Part D*

9—Part E

10—calf skin collagen and

FIGS. 3A and 3B are SDS-PAGE gels showing abalone collagen and calf skin collagen in which FIG. 3A has the following lanes:

Lane 1—molecular weight standard

2—calf skin collagen

3—calf skin collagen

4—1^{st} Extract.

5—1^{st} Extract incubated at 27° C. for 17 hrs

6—1^{st} Extract incubated at 27° C. for 48 hrs; and

FIG. 3B has the following lanes:

Lane 1—molecular weight standard

2—calf skin collagen

3-2^{nd} Extract; and

FIG. 4 is an SDS-PAGE gel of abalone gelatin in which lane 1 is a molecular weight standard, lane 2 is the gelatin and lane 3 is collagen 1^{st} extract.

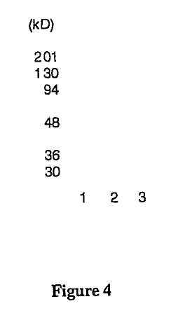

### MODES FOR CARRYING OUT THE INVENTION

Unless otherwise stated, all steps were carried out at 4° C. or on ice. All solvents and water used were pre-chilled at 4° C. This minimizes bacterial growth, enhances the solubility of native collagen, and ensures the retention of native conformation on the part of the solubilised collagen.

### EXAMPLE 1

Novel Method for Removal of Pigmentation from Abalone Tissue

The abalone foot is covered by skin where the mucus-secreting glands are located. The skin also contains cells that give colour. The colour varies with species type. The black-lip has black pigmentation. Currently abalone food processors have difficulty in the removal of pigment from abalone foot without breaking the meat up. The process used in the abalone food industry involves the forcing of a jet of warm water through a rumbler containing the abalone in order to remove the pigment, however this process is likely to convert collagen to gelatin thereby softening the meat and breaking it into pieces. This also changes the texture of the meat. A collagen molecule is transformed into gelatin by heat denaturation above body temperature.

It is highly preferable that the native collagen product described above be white, with absence of any black pigment. A process to remove the pigmentation without any thermal denaturation to the collagen is described in detail below, by way of example only.

Step 1. Abalone Fishing, Storage and Transport.

Tank West Coast Abalone (TWC)

Black-lip abalone (Haliotis ruber) was fished from Port Davey on the west coast of Tasmania. Around 2500 kg were collected on this particular trip. During the trip the animals were stored in crates measuring 1200 mm long×900 mm wide×300 mm deep, and holding 130 kg of abalone. The crates were stacked one on top of another in large ‘wet wells’ containing filtered seawater which is pumped through the crates from below. The transport time in the crates was 2 days.

Upon arrival at the process plant in Tasmania the animals were transferred to live holding tanks for two days at a temperature of 15-17° C. One type of tank measured 1040 mm long X. 500 mm deep and held 45-50 kg of abalone while the other measured 1090 mm long×600 mm deep and held 60-70 kg of abalone.

If there are no animals in the tanks for more than 3 days then it is dosed with salt. Sodium nitrate (1.5 g) and ammonium chloride (6 g) are dissolved in pits of water and pumped firstly through a biofilter, then through a bed of crushed abalone shells, into the tanks. So far this year the tanks have been dosed in Jan-Feb and May. The water is drained from the tanks between processes.

Five live abalone were air-freighted in March 2001 from Tasmania to Brisbane, Queensland. The abalone were transported from Tasmania to Queensland as a dry consignment. The abalone were placed in sealed, oxygen filled bags with wet foam to keep the humidity high. The animals were held vertically in a head down position by attachment to waxed cardboard sheets. This allows waste products to flow away from the animal. At all times during transport the animals were kept in an insulated container to maintain a constant temperature of 4° C. Upon arrival the abalone will have lost about 15% in body weight due to water loss. If they are returned to tanks promptly, they will regain this weight within 2-3 hours. It is not uncommon for abalone transported dry to arrive alive but to languish once returned to seawater.

Step 2. Shucking and Method of Tissue Preparation.

On arrival, only 2 or 3 abalone were still alive. All animals were shucked immediately. The animal was first washed under running water to remove any slime and sand. The animal was turned upside down and shucked by sliding a broad spatula under the foot at the flat region of the shell until the attachment of the foot to the shell was cut. Care was taken not to rupture any internal organs. The spatula was then run gently around the inside edge of the shell to detach the internal organs. The whole animal was then able to be tipped out of the shell.

The guts and other organs (visceral part) were carefully separated from the foot using a scalpel. Care was taken not to rupture any internal organs so as to avoid contamination of the foot tissue. The internal organs were further dissected, bagged separately and stored at −20° C. for other protein extraction. The mouth area was cut away from the front of the foot with a scalpel, bagged and stored at −20° C.

The foot was rinsed with water and weighed. Several deep incisions were made in the front area of foot with a scalpel and the foot suspended over a strainer to allow the blood to drain to a collection vessel. Care was taken to avoid bacterial contamination. This was done at 4° C. with an initial collection after 1 hour and a further collection after 6 hours. The blood was used for the preparation of haemocyanin as described in our co-pending application entitled “Novel Haemocyanin”, the contents of which are incorporated herein by reference, and the remaining tissue for the extraction of collagen. Any remaining organic material was scraped from the inside of the shell, which was rinsed with water and left to dry at room temperature.

Step 3. The weight of the abalone muscle tissue was measured and found to be 100 gms.

Step 4. The tissue was soaked in 0.2M acetic acid overnight with slight agitation.

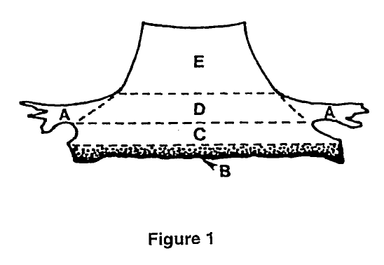

Step 5. The tissue was washed under running cold tap water which removed the pigmentation from the outer areas of the epipodoium, the hard part of the foot (pedal sole) and the upper part of the adductor (columellar) muscle (FIG. 1).

It will be appreciated that a simple and efficient process for the removal of pigment from the abalone foot area and adductor area has been developed. Soaking the tissue in 0.2 M acetic acid causes swelling and softens the tissue, thus allowing easy removal of black pigmentation from the outer regions of the foot (pedal sole) epidermis. Too much swelling is a disadvantage since it results in a rigid and stiff tissue.

This process will be of value to the abalone food processing industry as well as aiding in the extraction white collagen fibrils.

### EXAMPLE 2

Extraction of Native Collagen from the Individual Parts of the Muscle Tissue Using Acetic Acid

The presence, quantity and quality of collagen from the different parts of the abalone muscle were determined. The abalone muscle was divided into foot (pedal sole), the dorsal surface of foot (epipodium), and adductor (columellar) muscle (see FIG. 1). The foot and adductor muscle were further separated into soft and hard parts, and upper and middle parts, respectively.

Step 1. After removal of the pigments from the tissue the various muscle parts (A, B, C, D, and E) were dissected using a scalpel and the individual parts weighed:

Part A=4 gm

Part B=28 gm

Part C=7 gm

Part D=47 gm

Part E=3 gm

Each individual part was treated separately as follows.

Step 2. The tissue was further cut into smaller pieces using a scalpel.

Step 3. 0.5 M acetic acid solution (pH 3.0) was added to the tissue.

Part A=50 ml

Part B=100 ml

Part C=50 ml

Part D=200 ml

Part E=200 ml

Step 4. The individual suspensions (part A, B, C, and E) were stirred overnight.

Part D was not stirred and allowed to stand overnight. The supernatant (D*) was retained for analysis to determine if collagen was extracted without any agitation to the tissue. A further 200 ml of 0.5 M acetic acid was added to the remaining part D tissue.

Step 5. The suspensions were homogenised using a hand held blender.

Step 6. The pH of the slurry was adjusted to 3.5 with a small volume of 1.0 N HCl.

Step 7. The slurry was stirred overnight to extract collagen fibrils.

Step 8. The stirrer was turned off and the solids were permitted to settle out.

Step 9. The solution was centrifuged at 3,000 rpm, for 20 minutes to remove tissue particulates.

Step 10. In order to precipitate the native collagen fibrils the supernatant was brought to 0.3M sodium chloride by gradually adding solid sodium chloride to the supernatant with constant stirring. Visible white collagen fibrils precipitated within 2 minutes.

Step 11. The solution was allowed to stir overnight to further extract the native collagen fibrils.

Step 12. The solution had a high viscosity indicating the presence of collagen.

Step 13. The native collagen fibrils were collected by centrifugation at 5,000 rpm at 4° C. for 30 minutes.

Step 14. The native collagen fibrils from parts A, B, C, D, D* and E were each dissolved in a minimum quantity of de-ionised water.

Step 15. The native collagen fibrils were extensively dialysed against de-ionised water to remove any salt.

Step 16. The native collagen fibrils from parts A, B, C, D, D*, and E were transferred into separate freeze drying bottles and frozen in liquid nitrogen.

Step 17. The samples were freeze dried for approximately 16 hours.

Chemical Analysis

1. Protein Estimation

Protein estimation was carried out using the Pierce BCA assay. This method is based on the reduction in alkaline conditions of Cu²⁺ to Cu¹⁺ by protein (biuret reaction) and the colourimetric detection of Cu¹⁺ using bicinchoninic acid (BCA). An appropriate amount of working reagent was prepared by the mixture of 50 parts of reagent A and 1 part of reagent B. For each sample, 2 ml of working reagent was aliquoted into Johns 5 ml polystyrene tubes.

The freeze dried abalone collagen samples (A, B, C, D, D* and E) and calf skin collagen (Sigma Chemicals) were resuspended with de-ionised water to a concentration of 1 mg/ml. Then 0.1 ml of each sample was added to a tube and mixed by gentle inversion. A blank was prepared using 0.1 ml de-ionised water. The tubes were placed in a preheated water bath at 37° C. for 30 minutes, then allowed to cool on the bench for 10 minutes.

A standard curve was prepared by diluting a stock solution of BSA to a range of concentrations from 25-2000 μg/ml and assaying as described above.

The samples were read on a Biorad Smart Spec 3000 spectrophotometer using the inbuilt BCA protein assay function. This allows the storage of standard curves and automatic calculation of sample concentration. Disposable UV grade PMMA cuvettes were used for absorbance measurement at 562 nm.

2. Molecular Weight, Purity and Chain type Composition Determination

The molecular weight, purity and chain type composition of abalone collagen from each part (A, B, C, D, D* and E) was evaluated by sodium dodecyl sulphate polyacrylamide gel electrophoresis (SDS-PAGE). 8% Gradipore iGel precast Tris glycine gels was used. SDS-PAGE was performed according to the method of Laemmli (1970).

Freeze dried abalone collagen (samples A, B, C, D, D* and B) and calf skin collagen were dissolved at 1 mg/ml in deionised water. Samples were then diluted to half strength with Gradipore Glycine sample buffer.

The samples were then placed into a boiling water bath for 3 minutes, then allowed to cool. The gel was assembled in a Biorad Mini-Protean 3 electrophoresis cell. The inner chamber was filled with SDS glycine running buffer and the samples loaded with an autopipettor and standard yellow tips. The total protein load per well was 2 μg. A molecular weight marker (Biorad broad range prestained marker) was run with each gel. The outer chamber was filled with running buffer to the level of the wells.

The running conditions were 150V constant voltage over 60 minutes with an approximate start current of 50 mA. The gel was then removed from the casing and rinsed with water for around 30 seconds. The gel was stained with around 50 ml of Gradipore Gradipure stain (based on colloidal G-250 Coomassie blue) overnight with gentle shaking. The gel was destained with frequent changes of water. Bands were generally visible after 5 minutes with about a day required for complete destaining.

Permanent storage of gels was achieved by drying between cellophane sheets. The destained gels were soaked in a drying solution of 20% methanol and 2°% glycerol with gentle shaking for 15 minutes. Two cellophane sheets per gel were wetted in the drying solution for around 30 seconds. The trimmed gel was clamped between the cellophane sheets in a drying frame and left to stand in an open container at room temperature for 2 days. The gel was then pressed for a number of days prior to display.

3. Solubility Determination

The solubility of freeze dried material from parts A, B, C, D, D and E were tested.

Test Method:

1. To around 10 mg of each freeze dried sample, de-ionised water was added to 1 mg/ml and swirled.

1. 20 μl of 1M HCl was added with moderate swirling.

2. The tubes were left on their side for gentle swirling on an orbital shaker, then stood upright and allowed to settle. The clarity of the solution was observed.

Results

Table 1 shows the total Weight of Freeze Dried Native Abalone Collagen Fibrils (Parts A, B, C, D, D* and E) and Their Appearance.

TABLE 1

| | Total freeze dried | |
|---|---|---|
| Sample | weight (g) | Appearance |
| A | 0.389 | White, spongy |
| | | fibres |
| B | 1.98 | White, spongy |
| | | fibres |
| C | 6.34 | White, crisp, |
| | | dense fibres |
| D | 1.43 | White, spongy |
| | | fibres |
| D* | 3.19 | Cream, rubbery |
| E | 1.8 | White, crisp |
| | | fibres |

Table 2 shows Native Abalone Collagen Fibril Extraction Yield.

TABLE 2

| Sample | Yield (%) |
|---|---|
| A | 10 |
| B | 7.1 |
| C | 91 |
| D | 3.1 |
| D* | 6.8 |
| E | 60 |

Table 3 shows Protein Content of Native Abalone Collagen Fibrils (Part A, B, C, D, D* and E).

TABLE 3

| | Protein Concentration by |
|---|---|
| | BCA |
| Sample | (mg/ml) |
| Part A | 0.430 |
| Part B | 0.449 |
| Part C | 0.428 |
| Part D | 0.421 |
| Part D* | 0.073 |
| Part E | 0.636 |
| Calf skin collagen | 0.484 |

Table 4 shows the Solubility of Native Abalone Collagen Fibrils (Parts A, B, C, D, D* and E)

TABLE 4

| Sample | Appearance |
|---|---|
| A | clear solution |
| B | Clear solution |
| C | Clear solution |
| D | Clear solution |
| D* | Large amount settled and suspended |
| | material |
| E | clear solution |

A large amount of collagen could be extracted from the different parts of the abalone tissue when treated with 0.5 M acetic acid. When the collagen fibrils in a tissue are treated with 0.5 M acetic acid at pH 3.5 the hydrolysis of unstable cross-links releases into solution ‘acid-soluble’ native collagen.

Approximately 91° of native abalone collagen was extracted from muscle part C and 60% from part E, while part A, B, and D and D* were 10%, 7.1%, 3.1% and 6.8% respectively. Thus abalone contains large amounts of collagen in the muscle, which vary depending on muscle parts.

Analysis of D* sample indicated that 7% protein was extracted from non-homogenised and non-agitated raw material (Part D). However the freeze dried material from D* was cream coloured and rubbery with poor solubility as compared with the Part D sample which was white. This could be due to soaking the D sample in 0.5 M acetic acid overnight.

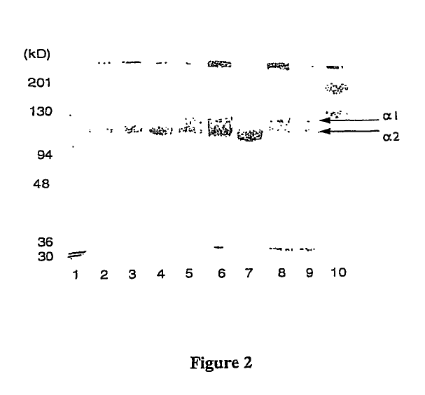

When examined by SDS-PAGE (FIG. 2) parts A, B, C, D* and E contained two major bands at 123.9 kD and 110.6 kD. These bands could be the α1 and α2 chains. Part D had just one single broad band at 105 kD. The molecular weight of native abalone collagen was significantly different from calf skin collagen which showed two main bands at 204 kD and at 138.5 kD.

The electrophoretic behaviour of abalone collagen has clearly demonstrated the occurrence of two types of alpha chain. Thus abalone native collagen is Type I, being the main protein constituent of abalone muscle tissue. The ratio of α1 and α2 in the different parts varied. In parts A and B there was a higher level of α2 than α1 chains. In parts C, D and E there were equal amounts of α1 and α2 chains. Part D had just one single broad band which is currently being analysed to determine if this protein is collagenous or non-collagenous.

### EXAMPLE 3A

Extraction of Native Abalone Collagen Fibrils from the Whole Muscle Tissue (1^{st} Extract)

Step 1. Abalone Fishing, Storage and Transport

Wild East Coast Abalone (WEC)

Black-lip abalone (Haliotis ruber) were fished from Storm Bay on the east coast of Tasmania. These animals were shipped directly to Brisbane, Queensland without tank storage at the abalone process plant in Tasmania.

Seven live abalone (batch 1) were air-freighted in March 2001 from Tasmania to Brisbane, Queensland (as described in Example 1 Step 1).

Step 2. Live Holding Tank

On arrival, the live animals were transferred to a holding tank. It measures 1430 mm long X 430 mm wide X 450 mm high, giving a volume of approximately 280 litres. A pump circulates the water through a filter and aeration system while a refrigeration unit controls the water temperature at 10° C.

The tank is sited in a separate room for quarantine purposes and is protected from fluctuations in the external environment. The status and movements of the animals were closely monitored and feeding of seafood pellets was conducted once a week. Abalone have been kept in the live holding tank for over a month with zero mortality. Water filtration is quite efficient and so the tank requires little cleaning.

Step 3. One abalone was removed from the tank after one day of storage.

Step 4. The total weight of the live abalone including the shell was weighed=450 gm.

Step 5. Shucking and Method of Tissue Preparation. The method is as described above for Example 1 Step 2.

Step 6. The weight of the abalone muscle tissue was measured (146 gm).

Step 7. The pigmentation from the foot area and adductor area was removed as described in Example 1 (Steps 4-5).

Step 8. The muscle tissue was re-weighed (127 gm).

Step 9. The whole muscle tissue was cut into smaller pieces using a scalpel.

Step 10. 1000 ml of 0.5 M acetic acid solution (pH 3.0) was added to the tissue.

Step 11. The mixture was stirred for 2 hours.

Step 12. The mixture was further homogenised using a hand held blender.

Step 13. The pH of the slurry was adjusted to 3.5 with a small volume of 1.0 N HCl.

Step 14. The slurry swelled and therefore another 500 ml of 0.5 M acetic acid solution (pH 3.0) was added.

Step 15. The slurry was stirred overnight to extract native collagen fibrils.

Step 16. The mixture was centrifuged at 3,000 rpm, for 20 minutes to remove tissue particulates. The pelleted tissue was retained for further extraction.

Step 17. In order to precipitate the native collagen fibrils the supernatant was brought to 0.3M sodium chloride by gradually adding solid sodium chloride to the supernatant with constant stirring. Visible white collagen fibrils precipitated within 2 minutes.

Step 18. The mixture was allowed to stir overnight to further extract native collagen fibrils.

Step 19. The solution had a high viscosity indicating the presence of collagen.

Step 20. The native collagen fibrils were collected by centrifuging at 5,000 rpm at 4° C. for 30 minutes.

Step 21. Solid sodium chloride was added to 1250 ml of supernatant (2^{nd} extraction) to give a final concentration of 0.3 M.

Step 22. The solution was allowed to stir overnight.

Step 23. The solution was clear and not viscous.

Step 24. The solution was centrifuged at 5,000 rpm to pelletise the native collagen fibrils. Very little pellet was present in the second extraction.

Step 25. The collagen pellets obtained from Step 20 and Step 24 were pooled.

Step 26. The native collagen fibrils were dissolved in a minimum quantity of de-ionised water.

Step 27. The native collagen fibrils were extensively dialysed against de-ionised water to remove salt.

Step 28. The native collagen fibrils were then dialysed against 0.1 M acetic acid. The dialysis medium was replaced frequently by fresh acid until the pH of the solution inside the dialysis bag reached 3.5.

Step 29. The native collagen fibrils were transferred into freeze drying bottles and frozen in liquid nitrogen.

Step 30. The sample was freeze dried for approximately 16 hours.

Step 31. The freeze dried collagen samples were weighed.

### EXAMPLE 3B

Re-Extraction of Collagen Fibrils (2^{nd} Extract) from 1^{st} Extract Pellet Tissue.

Step 1. The pellet (110 gm) obtained in Step 16 of Example 3A was re-extracted with 1500 ml of 0.5 M acetic acid.

Step 2. The mixture was stirred overnight to extract native collagen fibrils.

Step 3. The mixture was centrifuged at 5,000 rpm, at 4° C. for 20 minutes to remove tissue particulates.

Step 4. Solid sodium chloride was added gradually to the 1480 ml of the supernatant with constant stirring to give a final concentration of 0.3 M.

Step 5. The solution was allowed to stir overnight to precipitate native collagen fibrils.

Step 6. The solution did not have a high viscosity.

Step 7. The solution was centrifuged at 5,000 rpm at 4° C. for 30 minutes.

Step 8. The native collagen fibrils were dissolved in a minimum quantity of de-ionised water.

Step 9. The native collagen fibrils were extensively dialysed against de-ionised water to remove salt.

Step 10. Then the native collagen fibrils were dialysed against 0.1 M acetic acid until the pH of the solution inside the dialysis bag reached pH 3.5.

Step 11. The collagen sample was transferred into freeze drying bottles, frozen in liquid nitrogen and freeze dried for 16 hours.

Step 12. The freeze dried collagen samples were weighed.

Chemical Analysis

1. Protein Estimation

Protein estimation was carried out using the Pierce BCA assay. This method is based on the reduction in alkaline conditions of Cu²⁺ to Cu¹⁺ by protein (buiret reaction) and the colourimetric detection of Cu¹⁺ using bicinchoninic acid (BCA). An appropriate amount of working reagent was prepared by the mixture of 50 parts of reagent A and 1 part of reagent B. For each sample, 2 ml of working reagent was aliquoted into Johns 5 ml polystyrene tubes.

The freeze dried abalone collagen samples (1^{st} and 2^{nd} extracts) and Sigma Calf Skin collagen were resuspended with de-ionised water to a concentration of 1 mg/ml. Then 0.1 ml of each sample was added to a tube and mixed by gentle inversion. A blank was prepared using 0.1 ml de-ionised water. The tubes were placed in a preheated water bath at 37° C. for 30 minutes, then allowed to cool on the bench for 10 minutes.

A standard curve was prepared by diluting a stock solution of BSA to a range of concentrations from 25-2000 μg/ml and assaying as described above.

The samples were read on a Biorad Smart Spec 3000 spectrophotometer using the inbuilt BCA protein assay function. This allows the storage of standard curves and automatic calculation of sample concentration. Disposable UV grade PMMA cuvettes were used for absorbance measurement at 562 nm.

2. Molecular Weight, Purity and Chain Type Composition Determination

The molecular weight, purity and type composition of abalone collagen (1^{st} and 2^{nd} extracts) were evaluated by sodium dodecyl sulphate polyacrylamide gel electrophoresis (SDS-PAGE). 12% (1^{st} extract) and 8% (2^{nd} extract) Gradipore iGel precast Tris glycine gels were used. SDS-PAGE was performed according to the method of Laemmli (1970).

Freeze dried abalone collagen samples (1^{st} and 2^{nd} extracts) and calf skin collagen were dissolved at 1 mg/ml in deionised water. Samples were then diluted to half strength with Gradipore Glycine sample buffer.

The samples were then placed into a boiling water bath for 3 minutes then allowed to cool. The gel was assembled in a Biorad Mini-Protean 3 electrophoresis cell. The inner chamber was filled with SDS glycine running buffer and the samples loaded with an autopipettor and standard yellow tips. The total protein load per well was 2 μg. A molecular weight marker (Biorad broad range prestained marker) was run with each gel. The outer chamber was filled with running buffer to the level of the wells.

The running conditions were 150 V constant voltage over 60 minutes with an approximate start current of 50 mA. The gel was then removed from the casing and rinsed with water for around 30 seconds. The gel was stained with around 50 ml of Gradipore Gradipure stain (based on colloidal G-250 Coomassie blue) overnight with gentle shaking. The gel was destained with frequent changes of water. Bands were generally visible after 5 minutes with about a day required for complete destaining.

Permanent storage of gels was achieved by drying between cellophane sheets. The destained gels were soaked in a drying solution of 20% methanol and 2% glycerol with gentle shaking for 15 minutes. Two cellophane sheets per gel were wetted in the drying solution for around 30 seconds. The trimmed gel was clamped between the cellophane sheets in a drying frame and left to stand in an open container at room temperature for 2 days. The gel was then pressed for a number of days prior to display.

3. Amino Acid Analysis

Amino acid analysis of collagen samples (abalone 1^{st} and 2^{nd} extracts and calf skin) was done using a Waters amino acid analyser. Samples containing ˜5 μg of protein were prepared for amino acid analysis from 1 mg/ml resuspensions of freeze dried collagen.

Calf Skin Collagen standard: (S1)

1^{st} Extract Abalone Collagen: (S2)

2^{nd} Extract Abalone Collagen: (S3).

4. Incubation of Collagen at 27° C.—Stability Trial

1^{st} Extract freeze dried native collagen was incubated in an oven at 27° C. Samples were taken after 17 and 48 hours and analysed by SDS-PAGE as described above.

5. Solubility Determination

The solubility of freeze dried material from the 1^{st} and 2^{nd} abalone collagen extracts and the calf skin collagen were tested.

Test Method:

1. To around 10 mg of both abalone freeze dried collagen samples de-ionised water was added to 1 mg/ml and swirled.

To around 1 mg of calf skin freeze dried collagen, de-ionised water was added to 1 mg/ml and swirled.

2. 10 μl of 1M HCl was added to the 1st extract and calf skin samples, with moderate swirling.

20 μl of 1M HCl was added to the 2^{nd} extract sample, with moderate swirling.

3. The tubes were left on their side for gentle swirling on an orbital shaker, then stood upright and allowed to settle. The clarity of the solution was observed.

Results

Table 5 shows the Total Weight of Freeze Dried Native Abalone Collagen Fibrils (1^{st} Extract and 2^{nd} Extract) and Calf Skin Collagen and Their Appearance.

TABLE 5

| | Total freeze dried | |
|---|---|---|
| Sample | weight (gm) | Appearance |
| 1^{st} Extract | 9.68 | white, crisp, |
| | | fibrous |
| 2^{nd} Extract | 2.79 | white fibrous, |
| | | spongy |
| Calf Skin | — | white, spongy |

Table 6 gives the Protein Content of Freeze Dried Native Abalone Collagen Fibrils (1^{st} Extract and 2^{nd} Extract) and Calf Skin Collagen.

TABLE 6

| | Protein Concentration by BCA |
|---|---|
| Sample | (mg/ml) |
| 1^{st} Extract | 0.621 |
| 2^{nd} Extract | 0.165 |
| calf skin collagen | 0.482 |

Table 7 describes the Solubility of Native Abalone Collagen Fibrils (1^{st} and 2^{nd} extracts) and Calf Skin Collagen.

TABLE 7

| Collagen Sample | Appearance |
|---|---|
| Abalone 1^{st} | solution clear |
| extract | |
| Abalone 2^{nd} | large amount of settled and suspended |
| extract | material |
| Calf skin | solution slightly turbid |

Table 8 gives the Amino Acid Composition of Native Abalone Collagen Fibrils (1^{st} and 2^{nd} Extracts) and Calf Skin Collagen.

TABLE 8

| Amino Acid | | | |
|---|---|---|---|
| Residue | S1 | S2 | S3 |
| Asp | 40.5 | 101.5 | 111.3 |
| Glu | 80.9 | 185.5 | 197.0 |
| Ser | 30.0 | 57.4 | 64.8 |
| Gly | 300.8 | 77.5 | 73.1 |
| His | 2.9 | 9.7 | 10.8 |
| Arg | 44.1 | 62.6 | 67.8 |
| Thr | 17.0 | 53.3 | 58.1 |
| Ala | 107.1 | 82.8 | 90.4 |
| Pro | 131.9 | 37.8 | 37.4 |
| Tyr | 5.4 | 21.9 | 22.7 |
| Val | 19.2 | 46.7 | 48.5 |
| Met | 5.5 | 24.7 | 20.1 |
| Ile | 8.8 | 31.8 | 33.8 |
| Leu | 56.4 | 124.3 | 134.8 |
| Phe | 15.0 | 27.5 | 25.8 |
| Lys | 22.6 | 54.5 | 62.3 |
| 4-OH-Pro | 142.5 | 11.5 | 6.0 |

S1 - calf skin collagen
S2 - 1^{st} extract abalone collagen
S3 - 2^{nd} extract abalone collagen

### EXAMPLE 3C

All extraction steps were repeated this time at room temperature. The native collagen fibrils were prepared and analysed as discussed in Example 3A, with essentially no difference in the results achieved.

Examples 3A to 3C show that native acid-soluble collagen fibrils are advantageously extracted with 0.5 M acetic acid and separated by sodium chloride precipitation from the supernatant. Extraction with 0.5 M acetic acid solubilised a large amount of the total collagen in contrast to vertebrate collagens which do not contain any acetic acid soluble collagen. Solubilising 1 kg of calf skin with pepsin only yields 0.025% collagen (Lauran et al 1980). Most of the collagen was extracted in the first extraction (1^{st} extract, Table 5).

Thus native solubilised collagen fibrils extracted from abalone muscle tissue lead to purified Type I native collagen. Furthermore, the solubility of the native abalone collagen samples produced a clear solution while the calf skin collagen (mammalian source) produced a turbid solution (Table 7).

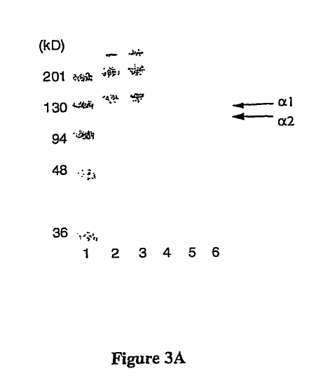

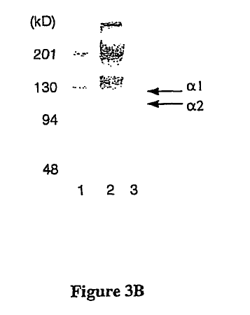

The SDS-PAGE gels exhibited two main bands at 123.9 and 110.6 kD (FIGS. 3A & 3B). The ratio of α1 and α2 chains of the 1^{st} and 2^{nd} extract were similar. Individual collagen chains were easily separated on the SDS-PAGE without column purification. Most type I collagens are composed of a heterotrimer of two α1(I) and α2 (I) chains which corresponds to the upper and lower chain bands respectively. The electrophoresis experiments conducted on calf skin collagen showed a main band at 204 kD (β chain) and bands at 138.5 and 132 kD, corresponding to α1 and α2 chains respectively (FIG. 3A).

Chemical composition and physical properties of fish meat show seasonal variations causing variations in texture (Dunajuski, 1979). Seasonal variations in free amino acids of seafood have also been reported on levels of glutamic acid, glycine, and taurine in lemon sole (Jones, 1959). Little study has been reported on whether chemical components in abalone meat show seasonal variations. The tough texture of abalone is considered important since abalone is usually consumed raw in Japan. During summer, abalone meat texture is not tough and this is related to the low collagen content. Thus customarily, summer is the best and winter is the worst season for abalone meat toughness.

The imino acids, proline and hydroxyproline are both stabilising factors, so that the melting temperature of collagen from many animals is proportional to the imino acid content (Jose and Harrington, 1964). The amino acid analysis of abalone native collagen fibrils is given in (Table 8). The hydroxyproline content of abalone collagen was low and this could be related to the seasonal catch as the abalone analysed in our work were summer abalone. There were variations in some of the residues between calf skin and abalone collagen, particularly a lower imino acid residue content in abalone collagen, indicating a lower denaturation temperature compared to calf skin collagen.

Glycosylation of hydroxylysine is related to extrusion of soluble collagen into the extracellular matrix. Large amounts of hydroxylysine residues may influence the structure of collagen fibrils (Blumenkrantz, 1969).

Collagen in the abalone meat may be important in energy storage and may have some effect on muscle metabolism before the spawning season, in order to make the gonads grow. An extraordinarily large growth of gonad index in abalone in spawning seasons has been reported (Webber 1970), thus abalone need much energy around spawning season. If abalone stored energy in muscle, storage of collagen might be reasonably expected because collagen is mainly composed of non-essential amino acids. Synthesis and decomposition of collagen might occur largely around spawning season. In summer such turnover might not be so active.

Following incubation at 27° C. for 17 and 48 hours, abalone native collagen fibrils had similar electrophoretic mobility as those stored at 4° C. (FIG. 3A). This indicates that abalone collagen has good thermal properties.

### COMPARATIVE EXAMPLE 1

Extraction of Abalone Collagen from the Whole Animal by Pepsin Digestion

Step 1. The pellet (wet tissue=110 gm) obtained in Step 16 of Example 3A was solubilised in 500 ml of 0.5 M acetic acid.

Step 2. To the solution was added 0.1 gm of pepsin (Sigma).

Step 3. The pH of the solution was adjusted to 2.8 with a small amount of 1 N HCl.

Step 4. The solution was stirred at room temperature for 8 hours then at 4° C. overnight for further extraction.

Step 5. The tissue was completely solubilised.

Step 6. The pH of the solution was changed from 2.8 to 6.0 with a small of amount of 1 M sodium hydroxide to stop the enzymatic action of the pepsin.

Step 7. The solution was centrifuged at 10,000 rpm at 4° C. for 1 hour.

Step 8. The collagen pellets were dissolved in a minimum quantity of de-ionised water and pooled.

Step 9. The collagen samples were transferred into freeze drying bottles, frozen in liquid nitrogen and freeze dried for 16 hours.

Step 10. The freeze dried collagen samples were weighed.

Chemical Analysis

1. Molecular Weight, Purity and Chain Type Composition Determination

The molecular weight, purity and type composition of pepsin-solubilised abalone collagen was evaluated by sodium dodecyl sulphate polyacrylamide gel electrophoresis (SDS-PAGE). 8% Gradipore iGel precast Tris glycine gels was used. SDS-PAGE was performed according to the method of Laemmli (1970).

Freeze dried pepsin-solubilised abalone collagen was dissolved at 1 mg/ml in deionised water. Samples were then diluted to half strength with Gradipore Glycine sample buffer.

The samples were then placed into a boiling water bath for 3 minutes then allowed to cool. The gel was assembled in a Biorad Mini-Protean 3 electrophoresis cell. The inner chamber was filled with SDS glycine running buffer and the samples loaded with an autopipettor and standard yellow tips. The total protein load per well was 2 μg. A molecular weight marker (Biorad broad range prestained marker) was run with each gel. The outer chamber was filled with running buffer to the level of the wells.

The running conditions were 150 V constant voltage over 60 minutes with an approximate start current of 50 mA. The gel was then removed from the casing and rinsed with water for around 30 seconds. The gel was stained with around 50 ml of Gradipore Gradipure stain (based on colloidal G-250 Coomassie blue) overnight with gentle shaking. The gel was destained with frequent changes of water. Bands were generally visible after 5 minutes with about a day required for complete destaining.

Permanent storage of gels was achieved by drying between cellophane sheets. The destained gels were soaked in a drying solution of 20% methanol and 2% glycerol with gentle shaking for 15 minutes. Two cellophane sheets per gel were wetted in the drying solution for around 30 seconds. The trimmed gel was clamped between the cellophane sheets in a drying frame and left to stand in an open container at room temperature for 2 days. The gel was then pressed for a number of days prior to display.

2. Solubility Test

Test Method:

1. To around 10 mg of freeze dried sample, add de-ionised water to 1 mg/ml and swirl for 15 minutes.

2. Add 20 μl of 1M HCl and give moderate swirling for around 2 hours.

3. The tubes were left on their side for gentle swirling on an orbital shaker, then stood upright and allowed to settle. The clarity of the solution was observed.

Results

Table 9 shows the Total Weight of Freeze Dried Pepsin-Solubilised Abalone Collagen and Its Appearance.

TABLE 9

| | Total freeze | |
|---|---|---|
| Sample | dried weight (g) | Appearance |
| Pepsin-solubilised | 0.878 | Yellow/cream |
| collagen | | colour |
| | | Spongy |

Due to the lack of solubility of the pepsin-solubilised collagen, the collagen bands were not visible on the SDS-PAGE gel (gel not shown). Therefore, analysis of the molecular weight, purity and chain type composition did not prove to be possible.

Table 10 gives the Solubility of Native Abalone Collagen Fibrils (pepsin-solubilised collagen).

TABLE 10

| Sample | Appearance |
|---|---|
| pepsin-solubilised | cloudy/turbid, with very small |
| collagen | suspended flakes |

In contrast to the process of the invention, the use of pepsin to solubilise abalone muscle tissue produces a yellow coloured final product. It will not be cost-effective to use this process on an industrial scale as pepsin is an expensive agent and further more the final product does not retain the native structure of collagen. The poor solubility of the freeze dried sample could be due to prolonged freeze drying. Solubilisation of pepsin-digested collagen results from hydrolysis of peptide bonds within the telopeptides between the cross-linking sites and the triple helix. Nevertheless, abalone is a hitherto unexpected source of collagen as discussed in our co-pending patent application entitled “Novel Process”, the disclosure of which is incorporated herein by reference.

### EXAMPLE 5

Isolation of Gelatin

Step 1. The pigment from the abalone tissue was removed as described in Example 1 Steps 4-5.

Step 2. The tissue (50 gms) was homogenised and to the slurry was added 200 ml 0.5 M acetic acid (pH 3.5) to extract the gelatin. The extraction was carried out in a water bath at 40° C.

Step 3. The slurry was centrifuged at 3,000 rpm for 30 minutes, 25° C. to remove tissue particles.

Step 4. The gelatin solution was transferred into a freeze drying bottle, frozen in liquid nitrogen and freeze dried for 16 hours.

Step 5. The freeze dried gelatin sample was weighed.

Chemical Analysis

Method

1. Molecular Weight and Purity

The abalone gelatin sample was analysed as discussed in Example 3A. A native abalone collagen sample was also included for comparison.

2. Solubility

The freeze dried material was dissolved at 1 mg/ml in de-ionised water as discussed in the collagen method section (Example 3A).

3. Gelling Properties of Abalone Gelatin

2 ml of abalone gelatin was left at room temperature to observe the gelling properties.

Results

Table 11 shows Total Weight of Freeze Dried Abalone Gelatin and Its Appearance

TABLE 11

| | Total freeze dried | |
|---|---|---|
| Sample | weight (g) | Appearance |
| gelatin | 1.65 | white, fibrous and |
| | | spongy |

Table 12 gives Solubility of Abalone Gelatin

TABLE 12

| Sample | Appearance |
|---|---|
| gelatin | clear solution |

The abalone gelatin had a molecular weight of 110 kD on SDS-PAGE (FIG. 5) and exhibited good solubility (Table 12).

The abalone gelatin gelled at room temperature in a matter of minutes which shows good gelling properties compared to calf skin gelatin which would require 4° C. overnight in order to set.

Thus, there has been developed a novel, simple and cost effective process for the extraction of gelatin from abalone waste tissue. Alternatively gelatin could be prepared from isolated collagen by heating, as would be well understood by the person skilled in the art.

### Industrial Applicability

Native Type I collagen is useful in the following areas:

cosmetic ingredients

injectable collagen

biomedical devices (cell growth matrices, prosthetic devices, synthetic skin, and dressings for wounds)

pharmaceutical

food (eg ingredient in sausages)

beverage (fining agent)

culture media and generally as a replacement for collagen prepared from land vertebrates by conventional techniques.

Gelatin is useful at least in the following areas:

Food industry (edible gelatin, flocculating agent in beverages)

industrial gelatin (manufacture of PVC pipes, glue, and carbonless paper)

Photographic gelatin (for emulsion formation)

Pharmaceutical (capsule coating)

Cosmetics (due to its hypo-allergenic and hydrating properties)

The unique characteristics which give gelatin wide application in industry are its abilities to gel, thicken, stabilise, emulsify, bind, film and aerate.

The gelatin of the invention is useful generally as a substitute for gelatin from conventional sources prepared by conventional techniques.

### REFERENCES

The references listed below have their disclosure incorporated herein through this reference:

Blumenkrantz N., Rosebloom J. and Prockop D. J. (1969) ibid 192, 81-89

Dunajuski E. (1979) J Texture Studies 10, 301-308

Francois C. J. and Glincher M. J. (1967) Biochim. Biophys. Acta 133, 91.

Helseth D. L Jr and Veis A (1981) J. Biol. Chem. 256, 7118-7128.

Hofmann H, Fietzek, P. P and Kuhn K (1980) J. Mol Biol. 141, 293-314.

Jose J, and Harrington W. F (1964) J. mol Biol 9, 269-287.

Katzman R. L and Kang A. H (1972) J. Biol. Chem 247, 5486.

Kefalides N. A (1971) Biochem Biophys Res. Commun. 46, 226.

Laemmil U. K (1970) Nature 227, 680-685.

Laurain G, Delvincourt T, and Szymanowicz A. G. (1980) FEBS Letter, 120, 44-48.

Lewis M. S and Piez K. A. J. (1964) Biol. Chem. 239, 336.

McClain P. E., Creed G. J., Wiley E. R. and Gerrits R. J. (1970) Biochim. Biophys Acta 221, 349.

Miller E. J Biochemistry (1971) 10, 1652.

Miller E. J., Martin G. R., Piez K—A and Powers M. J. J. Biol. Chem (1967) 242, 5481.

Piez K. A Biochemistry (1965) 4, 2590.

Piez, K. A, Eiger A, and Lewis M. S (1963) Biochemistry 2, 58.

Piez K. A (1984) Molecular and aggregate structures of the collagens. In Extracellular Matrix Biochemistry (Piez, K. A and Reddi A. H. eds) pp 1-39, Elsevier New York.

Prockop D. J and Kivirikko K. I (1995) Annu. Rev. Biochem 64, 403-434.

Reiser K. M (1991) Proc. Soc. Exp. Biol. Med. 196, 17.

Trelstad R. I. Kang A. H Igarashi S. and Gross J. (1970) Biochemistry 9, 4993.

Veis A. (1964) The Gelatin→Collagen Transition. Ch. 5 in The Macromolecular Chemistry of Gelatin, Academic Press, New York and London, pp 267-416

Webber H. H (1970) Physiol. Zool. 43, 213-231

1. A process for isolating a collagen-derived protein fraction from a marine invertebrate, comprising the steps of:
1) preparing a collagen-containing portion of said marine invertebrate for extraction;
2) treating the collagen-containing portion with a weak acid solution in order to solubilise a collagen-derived protein fraction; and
3) collecting the collagen-derived protein fraction.

2. A process as claimed in claim 1 wherein the weak acid solution is an acetic acid solution.

3. A process as claimed in claim 2 wherein the acetic acid solution has a concentration between 0.1M and 1M.

4. A process as claimed in claim 3 wherein the acetic acid solution is a 0.5M solution.

5. A process as claimed in any one of claims 1 to 4 wherein the temperature is maintained below that at which collagen converts to gelatin, and native collagen is collected.

6. A process as claimed in claim 5 wherein the temperature is maintained below 25° C.

7. A process as claimed in claim 5 wherein the temperature is maintained below 4° C.

8. A process as claimed in any one of claims 5 to 7 further comprising the step of adjusting the pH of the native collagen-containing weak acid solution.

9. A process as claimed in claim 8 wherein the pH is reduced by adding a strong acid.

10. A process as claimed in claim 9 wherein the pH is reduced by adding a 1.0N hydrochloric acid solution.

11. A process as claimed in claim 10 wherein the pH is adjusted to about 3.5.

12. A process as claimed in claim 11 wherein the pH adjustment takes place after the collagen-containing portion has been in contact with the weak acid solution for 2 to 12 hours.

13. A process as claimed in claim 12 wherein the native collagen is collected after a further period of 6 to 24 hours of contact between the collagen-containing portion and the weak acid solution.

14. A process as claimed in claim 13 wherein there is an additional period of contact between the collagen-containing material and the weak acid solution after collection of the native collagen, followed by collection of additional native collagen.

15. A process as claimed in any one of claims 5 to 14 wherein the native collagen is precipitated by salting out.

16. A process as claimed in claim 15 wherein the collagen-containing portion is spun down in a centrifuge and the native collagen is precipitated from the supernatant.

17. A process as claimed in claim 16 wherein the native collagen is collected through the addition of sodium chloride to the supernatant.

18. A process as claimed in claim 17 wherein sufficient sodium chloride is added to bring the supernatant to 0.3M sodium chloride.

19. A process as claimed in any one of claims 15 to 18 wherein the native collagen is dialysised against de-ionised water after it precipitates in order to remove the salt.

20. A process as claimed in claim 19 wherein the native collagen is then dialysised against a weak acid solution to adjust the pH.

21. A process as claimed in any one of claims 5 to 20, further comprising the step of freeze drying the purified native collagen.

22. A process as claimed in any one of claims 1 to 4 wherein the temperature equals or exceeds that at which collagen converts to gelatin, and gelatin is collected.

23. A process as claimed in claim 22 wherein the temperature is maintained at or at above 40° C.

24. A process as claimed in any one of claims 1 to 23 wherein the collagen-containing portion is suspended in the weak acid solution.

25. A process as claimed in claim 24 wherein the suspension is subjected to agitation.

26. A process as claimed in claim 25 wherein the suspension is stirred.

27. A process as claimed in any one of claims 1 to 26 wherein the marine invertebrate is prepared for extraction by mechanical disruption of the collagen-containing portion.

28. A process as claimed in any one of claims 1 to 27 wherein the collagen-containing portion is muscle tissue.

29. A process as claimed in claim 27 wherein pigment is removed from the intact muscle tissue.

30. A process as claimed in claim 28 wherein the intact muscle tissue is soaked in a weak acid solution.

31. A process as claimed in claim 29 wherein the weak acid solution is an acetic acid solution.

32. A process as claimed in claim 31 wherein the acetic acid solution is a 0.2% solution.

33. A process as claimed in any one of claims of claims 1 to 32 wherein the marine invertebrate is abalone.

34. A process as claimed in claim 33 wherein the marine invertebrate is selected from the group consisting of the black-lip abalone, Haliotis ruber, the brown-lip abalone, Haliotis conicopora, the green-lip abalone, Haliotis laevigata and Roe's abalone, Haliotis roei.

35. A process for obtaining native collagen, comprising the steps of:
1) providing a marine invertebrate from which collagen may be extracted;
2) preparing a collagen-containing portion of the marine invertebrate for extraction;
3) treating the collagen-containing portion with a weak acid solution in order to solubilise a collagen fraction; and
4) collecting the native collagen.

36. A process as claimed in claim 35 wherein the weak acid solution is an acetic acid solution.

37. A process as claimed in claim 36 wherein the acetic acid solution has a concentration between 0.1M and 1M.

38. A process as claimed in claim 37 wherein the acetic acid solution is a 0.5M solution.

39. A process as claimed in any one of claims 35 to 38 further comprising the step of adjusting the pH of the collagen-containing weak acid solution.

40. A process as claimed in claim 39 wherein the pH is reduced by adding a strong acid.

41. A process as claimed in claim 40 wherein the pH is reduced by adding a 1.0N hydrochloric acid solution.

42. A process as claimed in claim 41 wherein the pH is adjusted to about 3.5.

43. A process as claimed in claim 42 wherein the pH adjustment takes place after the collagen-containing portion has been in contact with the weak acid solution for 2 to 12 hours.

44. A process as claimed in claim 43 wherein the native collagen is collected after a further period of 6 to 24 hours of contact between the collagen-containing portion and the weak acid solution.

45. A process as claimed in claim 44 wherein there is an additional period of contact between the collagen-containing material and the weak acid solution after collection of the collagen, followed by collection of additional collagen.

46. A process as claimed in any one of claims 35 to 45 wherein the collagen-containing portion is suspended in a weak acid solution.

47. A process as claimed in claim 46 wherein the suspension is subjected to agitation.

48. A process as claimed in claim 47 wherein the suspension is stirred.

49. A process as claimed in any one of claims 35 to 48 wherein the native collagen is precipitated by salting out.

50. A process as claimed in claim 49 wherein the collagen-containing portion is spun down in a centrifuge and the native collagen is precipitated from the supernatant.

51. A process as claimed in claim 50 wherein the native collagen is collected through the addition of sodium chloride to the supernatant.

52. A process as claimed in claim 51 wherein sufficient sodium chloride is added to bring the supernatant to 0.3M sodium chloride

53. A process as claimed in any one of claims 49 to 52 wherein the native collagen is dialysised against de-ionised water after it precipitates in order to remove the salt.

54. A process as claimed in claim 53 wherein the native collagen is then dialysed against a weak acid solution to adjust the pH.

55. A process as claimed in any one of claims 35 to 54, further comprising the step of freeze drying the collagen.

56. A process as claimed in any one of claims 35 to 55 wherein the marine invertebrate is prepared for extraction by mechanical disruption of the collagen-containing portion.

57. A process as claimed in claim 56 wherein the collagen-containing portion is muscle tissue.

58. A process as claimed in claim 57 wherein pigment is removed from the intact muscle tissue.

59. A process as claimed in claim 58 wherein the intact muscle tissue is soaked in a weak acid solution.

60. A process as claimed in claim 59 wherein the weak acid solution is acetic acid solution.

61. A process as claimed in claim 60 wherein the acetic acid solution is a 0.2M solution.

62. A process as claimed in any one of claims of claims 35 to 61 wherein the marine invertebrate is abalone.

63. A process as claimed in claim 62 wherein the marine invertebrate is the black-lip abalone, Haliotis ruber, the brown-lip abalone, Haliotis conicopora, the green-lip abalone, Haliotis laevigata or Roe's abalone, Haliotis roei.

64. The product of the process of any one of claims 1 to 34.

65. The product of the process of any one of claims 35 to 63.

66. A process of de-pigmenting a marine invertebrate having undesirable pigmentation, comprising the steps of:
1) isolating the food portion of the marine invertebrate; and
2) contacting the food portion with a weak acid solution in order to extract the pigment.

67. A process as claimed in claim 66 wherein the food portion is the muscle tissue of the marine invertebrate.

68. A process as claimed in claim 67 wherein the marine invertebrate is abalone.

69. A process as claimed in any one of claims 66 to 68 wherein the food portion is soaked in the weak acid solution.

70. A process as claimed in claim 69 wherein the weak acid solution is an acetic acid solution.

71. A process as claimed in claim 70 wherein the acetic acid solution is a 0.2M solution.

72. A marine invertebrate when de-pigmented by the process of any one of claims 66 to 71.

73. An isolated polypeptide, said polypeptide being the α1 chain of type I abalone collagen, said polypeptide having a molecular weight of approximately 123.9 kD.

74. An isolated polypeptide, said polypeptide being the α2 chain of type I abalone collagen, said polypeptide having a molecular weight of approximately 110.6 kD.

75. An isolated polypeptide, said polypeptide being type I abalone collagen, comprising at least one α1 chain as defined in claim 73 and at least one α2 chain as defined in claim 74, and having the amino acid composition laid out in Table 8.

76. Native collagen isolated from abalone.

77. The use of a product as claimed in either one of claims 64 or 65 in place of collagen isolated from land vertebrates or gelatin prepared from the collagen of land vertebrates.

78. The use as claimed in claim 77 as cosmetic ingredients, injectable collagen, in biomedical devices, as a pharmaceutical substance, in food products and in beverages.

79. A cosmetic composition comprising a product as claimed in either one of claims 64 or 65 in addition to conventional cosmetic ingredients.

80. A biomedical device comprising collagen as claimed in either one of claims 64 or 65.

81. A biomedical device as claimed in claim 80 which is a cell growth matrix, prosthetic device, synthetic skin, a dressing for a wound.

82. A pharmaceutical composition comprising collagen as claimed in either one of claims 64 or 65 as a carrier and an active ingredient.

83. A food comprising a product as claimed in either one of claims 64 or 65 and food ingredients.

84. A beverage prepared using a product as claimed in either one of claims 64 or 65 as a fining agent.

85. A capsule for pharmaceutical use prepared from gelatin as claimed in either one of claims 64 or 65.

86. The use of collagen as claimed in claim 64 or 65 in the preparation of gelatin.

87. A process for preparing gelatin, comprising the steps of:
1) providing collagen as claimed in claim 64 or 65
2) heating the collagen to a temperature sufficient for conversion to gelatin to be effected.

88. A process as claimed in claim 87 wherein said collagen is heated to a temperature of at least 40° C.
